(19) United States
(12) Patent Application Publication
(10) Pub. No.: US 2022/0233140 A1
(43) Pub. Date: Jul. 28, 2022

(54) Remote Patient Monitoring System

(71) Applicant: Tricella Inc., Mountain View, CA (US)

(72) Inventor: Daniel M. Weng, Mountain View, CA (US)

(21) Appl. No.: 17/158,006

(22) Filed: Jan. 26, 2021

(51) Int. Cl. A61B 5/00; G16H 40/67; G16H 50/70; G16H 10/60; G16H 20/10; G16H 20/30; G16H 15/00; G16H 50/20; A61B 5/11; A61B 5/0205; A61B 5/145; A61B 5/1455; A61B 5/318

(52) U.S. Cl. CPC A61B 5/4833; A61B 5/022; G16H 50/70; G16H 10/60; G16H 20/10; G16H 20/30; G16H 15/00; G16H 50/20; A61B 5/1118; A61B 5/02055; A61B 5/14532; A61B 5/14551; A61B 5/318; A61B 5/743; A61B 5/7267; A61B 5/7221; G16H 40/67

(57) ABSTRACT

A health monitoring system provides information to healthcare professionals, patients, or caregivers of patients based on the correlation between adherence data and physiological data for the patient. The health monitoring system receives sensor data captured by a tracking device associated with a patient. Based on the sensor data, the health monitoring system generates adherence data. The adherence data includes a set of adherence data points, each corresponding to a time period of a set of time periods. Moreover, the health monitoring system receives physiological data captured by one or more measuring devices associated with the patient. The physiological data includes a set of physiological data points, each corresponding to a time period of the set of time periods. The health monitoring system then provides a user interface element generated based on a correlation between the generated adherence data and the physiological data to a user of the health monitoring system.

### BACKGROUND

### 1. Field of Art

This disclosure relates generally to a remote health monitoring system, and in particular for generating patient health information based on a correlation analysis between adherence data and physiological data for a patient of the remote health monitoring system.

### 2. Description of the Related Art

Human health is a complex field with multiple factors interacting with each other to produce certain responses in a person's body. Oftentimes, a person's health is characterized using a set of physiological measurements (such as blood pressure, blood glucose level, or heart rate). The set of physiological measurements can be tracked as a function of time to see how the physiological measurements evolve. However, understanding the changes in the physiological measurements for a patient and identifying the sources that significantly affect the physiological measurements for the patient can be challenging, particularly without quantifiable behavior data.

Moreover, if the physiological measurements are not within an expected or desired range, a physician can recommend a patient to start a prescription regimen. However, with a limited amount of information (especially in relations to adherence), it can be difficult for the physician to determine if the prescription regimen is working. Moreover, if the physiological measurements are not improving for a patient, the physician may not have enough information to determine if the prescribed therapeutics is not having the desired results, or other factors are preventing the prescribed therapeutics from providing the desired effect.

### SUMMARY

A health monitoring system provides information to healthcare professionals, patients, or caregivers of patients based on the correlation between (pharmaceutical and/or behavioral) adherence data and physiological data for the patient. The health monitoring system receives sensor data captured by a tracking device associated with a patient. Based on the sensor data, the health monitoring system generates adherence data. The adherence data includes a set of adherence data points, each corresponding to a time period of a set of time periods. Moreover, the health monitoring system receives physiological data captured by one or more measuring devices (such as a sphygmomanometer, a glucometer (or blood analysis device), a thermometer, a pulse oximeter, an electrocardiogram (ECG/EKG) monitor, or a breath analyzer) associated with the patient. The physiological data includes a set of physiological data points, each corresponding to a time period of the set of time periods. The health monitoring system then provides a user interface element generated based on a correlation between the generated adherence data and the physiological data to a user of the health monitoring system.

In some embodiments, the tracking device and the one or more measuring devices are configured to be connected to a client device of the patient. The tracking device sends the sensor data to the client device of the patient, and the one or more measuring devices send the recorded physiological data to the client device of the patient. The sensor data captured by the tracking device and the physiological data captured by the one or more measuring devices are then sent to the health monitoring system from the client device of the patient. In other embodiments, the tracking device or the one or more measuring devices are connected to a third-party system and the third-party system sends the sensor data or the physiological data to the health monitoring system (e.g., using an application programming interface). In yet other embodiments, the tracking device or the one or more measuring devices are directly connected to the health monitoring system via a computational module or network.

In some embodiments, the tracking device is a pillbox or medication dispenser having a set of sensors for determining whether a pill or medication by the pillbox or medication dispenser was accessed by the patient. For example, a pillbox includes a set of sensors for determining whether a compartment of the pillbox has been opened or accessed by the patient. In another example, a medication dispenser (such as a pill dispenser that stores and dispenses sealed packs containing one or more pills) includes one or more sensors for determining whether a medication pack was dispensed to the patient. In this embodiment, the adherence data is a drug adherence data indicating whether the patient consumed a prescribed medication within a series of set time windows.

In other embodiments, the tracking device is a fitness tracker for tracking the type of and amount of physical activity performed by the patient. In this embodiment, the adherence data is a physical activity adherence data indicating an amount of physical activity performed by the patient during each time period of the set of time periods.

In some embodiments, the user interface element is a graph for presenting the correlation between the generated adherence data and the physiological data for the patient. The graph overlays the adherence data with the physiological data. The graph is divided into a set of time periods, each displaying a corresponding adherence data point overlaid with a corresponding physiological data point. In other embodiments, the user interface element is a set of recommendations provided to a healthcare professional, nutritionist or fitness/wellness coaches associated with the patient. Each recommendation of the set of recommendations may be identified by applying a trained model to the generated adherence data and the physiological data for the patient. In yet other embodiments, the user interface element is a list of template messages for sending to the patient. Each template message of the list of template messages may be selected by applying a trained model to the generated adherence data and the physiological data for the patient.

In some embodiments, the user interface element is a list of patients associated with a healthcare professional. In this embodiment, the health monitoring system generates a relevance score for each patient based on a correlation analysis between the generated adherence data for the patient and the physiological data for the patient. The list of patients associated with the healthcare professional are then sorted based on the determined relevance score.

### BRIEF DESCRIPTION OF THE DRAWINGS

FIG. 1 is a block diagram of a system environment in which a health monitoring system operates, in accordance with one or more embodiments.

FIG. 2 is a block diagram of the health monitoring system, in accordance with one or more embodiments.

FIG. 3A illustrates an example of a pillbox and FIG. 3B illustrates the pillbox of FIG. 3A with a bin in the open position, in accordance with one or more embodiments.

FIG. 3C shows an exemplary sensor arrangement to detect an event in the pillbox having a bin in the closed position, in accordance with one or more embodiments.

FIG. 3D shows an exemplary sensor arrangement to detect an event in the pillbox having a bin in the opened position, in accordance with one or more embodiments.

FIG. 4A shows an example of a graph identifying whether a patient took a particular medicine pill, in accordance with one or more embodiments.

FIG. 4B shows an example graph plotting the physiological data as function of time, in accordance with one or more embodiments.

FIG. 4C shows an example graph plotting a patient's drug adherence data and physiological data combined into a single graph, in accordance with one or more embodiments.

FIG. 5 shows graphs overlaying a patient's physical activity adherence data with a patient's physiological data, in accordance with one or more embodiments.

FIG. 6A illustrates a user interface showing a list of patients for a healthcare professional in the health monitoring system, in accordance with one or more embodiments.

FIG. 6B illustrates a user interface showing details for a patient of the health monitoring system, in accordance with one or more embodiments.

FIG. 7 illustrates a flow diagram for employing patient's adherence data and patient's physiological data in the remote health monitoring of a patient, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### System Architecture

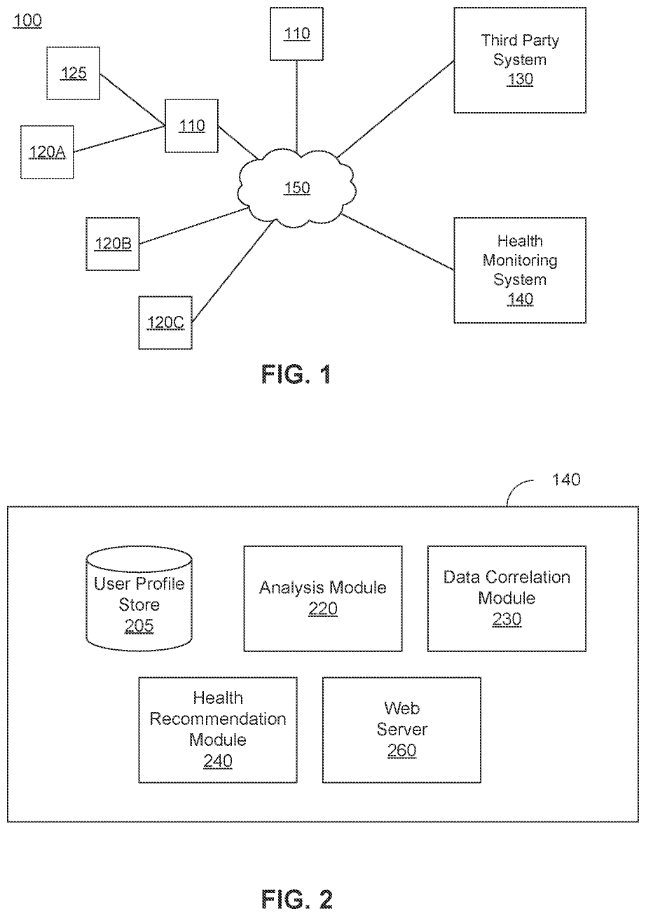

FIG. 1 is a block diagram of a system environment 100 for a health monitoring system 140, in accordance with one or more embodiments. The system environment 100 shown by FIG. 1 comprises one or more client devices 110, one or more physiological sensors or physiological measuring devices 120, one or more tracking devices 125 (such as a connected or smart pillbox and/or medication/supplement dispenser), one or more third-party systems 130, and the health monitoring system 140, and a network 150. In alternative configurations, different and/or additional components may be included in the system environment 100. For example, the health monitoring system 140 is a social networking system, a content sharing network, or another system providing content to users.

The client devices 110 are one or more computing devices capable of receiving user input as well as transmitting and/or receiving data via the network 150. In one embodiment, a client device 110 is a conventional computer system, such as a desktop or a laptop/portable computer. Alternatively, a client device 110 may be a device having computer functionality, such as a personal digital assistant (PDA), a data exchange hub, augmented reality accessory, a mobile telephone, a smartphone, or another suitable device. A client device 110 is configured to communicate via the network 150. In one embodiment, a client device 110 executes an application allowing a user of the client device 110 to interact with the health monitoring system 140. For example, a client device 110 executes a browser application to enable interaction between the client device 110 and the health monitoring system 140 via the network 150. In another embodiment, a client device 110 interacts with the health monitoring system 140 through an application programming interface (API) running on a native operating system of the client device 110, such as IOS® or ANDROID™. In yet other embodiments, the client device 110 interacts with the health monitoring system 140 through an API running through a platform aggregator module that can interface with various operating systems.

The client devices 110 are configured to communicate via the network 150, which may comprise any combination of local area and/or wide area networks, using both wired and/or wireless communication systems. In one embodiment, the network 150 uses standard communications technologies and/or protocols. For example, the network 150 includes communication links using technologies such as Ethernet, 802.11, worldwide interoperability for microwave access (WiMAX), 3G, 4G, 5G, narrow band internet of things (NBIOT) code division multiple access (CDMA), digital subscriber line (DSL), Sigfox, LORA, etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, all or some of the communication links of the network 150 may be encrypted using any suitable technique or techniques.

The measuring devices 120 are one or more devices that are capable of measuring physiological data of a patient. For example, the measuring devices 120 include a sphygmomanometer for measuring a patient's blood pressure, a glucometer for measuring a patient's blood sugar level, a blood analysis device for measuring cholesterol, lipids, ketone, uric acid, lactate and the like, a thermometer for measuring a patient's temperature, a pulse oximeter for measuring a patient's blood oxygen saturation, an electrocardiogram (ECG/EKG) monitor, and a breath analyzer, etc. A measuring devices 120 may be able to connect to the health monitoring system 140 via the network 150. In alternative embodiments, a measuring device 120 may be able to connect to a third-party system 130 (e.g., a system operated by a manufacturer of the measuring device 120) and the third-party system 130 provides the physiological measurements to the health monitoring system 140 upon request (e.g., via an application programming interface of API). In yet other embodiments, a measuring device 120 connects to a client device 110 (e.g., via Bluetooth or Wi-Fi). The measuring device 120 provides the physiological measurements to the client device 110, and the client device 110 provides the physiological measurements to the health monitoring system 140 via the network 150. In some embodiments, the client device 110 controls the measuring device 120. For example, the client device sends instructions to the measuring device 120 to start or stop taking measurements.

In some embodiments, the measuring devices 120 are capable of capturing or deriving neurological data for a patient. For example, the measuring devices 120 includes an electroencephalogram (EEG) monitor that captures neurological signals that can be used for identifying changes in mood, anxiety, or onset of psychosomatic episodes for a patient.

The tracking device 125 tracks one or more types of events for the patient. The tracking device 125 includes a set of sensors for tracking the one or more types of events. Different types of events that can be tracked by a tracking device 125 includes the consumption of a pill by a patient, the sleep pattern of a patient, the amount of exercise performed by the patient, and the like.

For example, the tracking device may be a pillbox. The pillbox 125 allows patients to store pills and monitors the patient's consumption of the pills. For instance, the pillbox 125 has sensors that monitor various events such as the opening and closing of the pillbox. For instance, the pillbox 125 has magnetic sensors that detect when a bin or a cover of the pillbox 125 has been opened or closed. The pillbox 125 transmits a signal (e.g., via a wireless communication protocol such as Bluetooth, 3G, 4G, 5G, Wi-Fi, NBIOT, Zigbee or a combination of) to a client device 110, the health monitoring system 140, or a third party system 130 in response to detecting an event (e.g., an event corresponding to the opening of a bin, or an event corresponding to the closing of a bin). For instance, as shown in the example environment of FIG. 1, the pillbox 125 wirelessly connects to the client device 110 of a patient. When the patient opens a bin of the pillbox 125 to access a pill stored in the bin, the sensor embedded in the pillbox senses the opening event and sends a notification to the client device 110. Moreover, when the patient closes the bin of the pillbox 125, the sensor embedded in the pillbox senses the closing event and sends a second notification to the client device 110. In some embodiments, the client device then sends a notification to the health monitoring system 140 regarding the opening or closing event for the pillbox, in addition to information identifying the patient within the health monitoring system (e.g., a username or a user identification number).

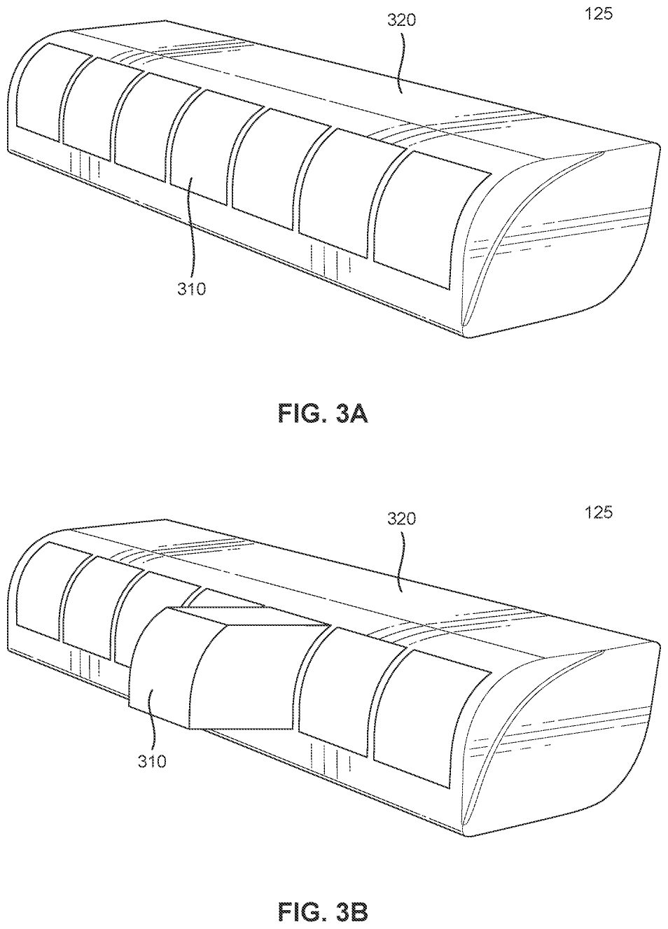

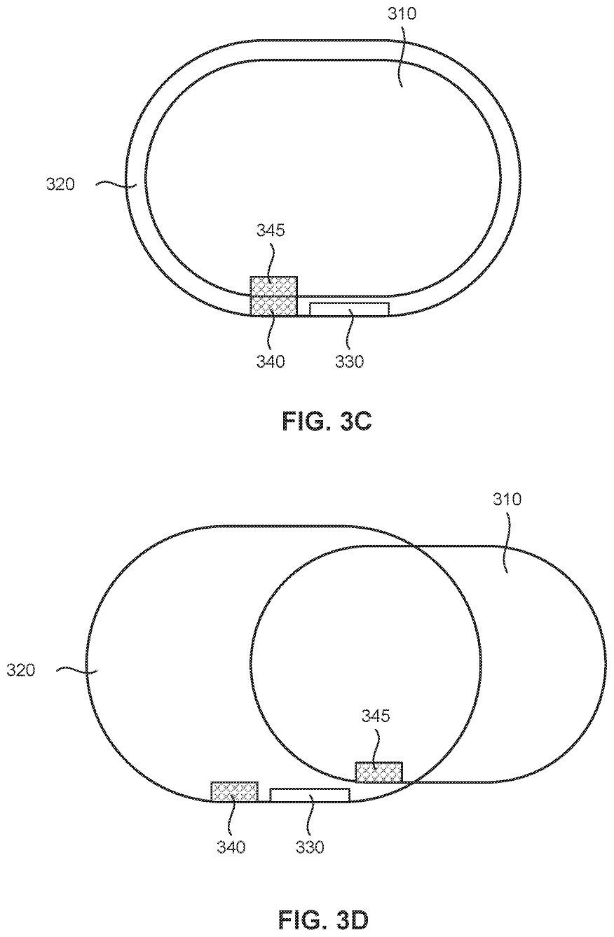

FIG. 3A illustrates an example of a pillbox and FIG. 3B illustrates the pillbox of FIG. 3A with a bin in the open position, in accordance with one or more embodiments. The pillbox 125 has multiple bins 310 or compartments for storing medicine pills. For example, the pillbox may have 7 bins, one for each day of a week. In another example, the pillbox 125 has four compartments, each compartment corresponding to one dose of a medicine to be taken four times a day (e.g., morning, afternoon, evening, and night).

In some embodiments, the pillbox additionally includes an outer housing 320. The outer housing holds the bins 310, and houses one or more sensors for detecting whether the bins are in an opened or in a closed position, and electronics for processing sensor data received from the one or more sensors and for communicating with a client device 110. In one embodiment, outer housing 320 has watertight seals (e.g., waterproof, or dish washer safe) on all the electrical components allows the pillbox 125 to be cleansed or rinsed with a liquid by the user without damage to its functionality or components.

In the example of FIG. 3B, the bins 310 are opened by sliding or translating the bins in a direction that is perpendicular to the outer housing. In some embodiments, the bins 310 may be pushed to open from an opposing side, which increases the ease of use for people with dexterity problems, e.g., arthritis patients. The bins 310 may have an elongated storage that allows contents (e.g., medicine pills) to be removed or dispensed. In one embodiment, each bin has markings to indicate timing information, e.g., day of the week or time of the day, or to indicate types of contents. The outer housing 320 may gave an oblong shape with rounding edges. Alternatively, the outer housing 320 has a different shape, e.g., lobe, or polygon, to prevent rolling on a sloped surface. The top and bottom of the outer housing 320 are flat to prevent rolling. The bottom of the outer housing can have an anti-skid surface to prevent unintentional sliding on a non-level surface as well as a sound dampening element for soft landing. In another example, the bin independently pivots or rotates to dispense contents.

FIG. 3C shows an exemplary sensor arrangement to detect an event in the pillbox having a bin in the closed position, in accordance with one or more embodiments. FIG. 3D shows an exemplary sensor arrangement to detect an event in the pillbox having a bin in the opened position, in accordance with one or more embodiments. In this example, component 345 is be a magnet or a conductive patch; accordingly, sensor 340 is a magnetic sensor or a conductive sensor with a corresponding PCB. In another embodiment, component 340 can also be other types of sensor (e.g., optical sensor, pressure sensor, volume sensor) with their corresponding PCBs. When bin 310 is in the closed position, as shown in FIG. 3C, the sensor 340 is able to detect the presence of component 345. For example, when the component 345 is a conductive or resistive path, the component 345 interacts with the sensor 340 to close a sensing circuit within the sensor when the component 345 is directly above the sensor 340. As such, the sensing circuit is closed when the bin is closed, and opened when the bin is opened.

In another example, when the component 345 is a magnet, the magnetic sensor 340 is able to detect the presence of the magnet. The magnetic sensor 340 may include an element that reacts to a magnetic field emitted by the magnet 345. When the magnetic sensor 340 is near the magnet 345, the element of the sensor moves in response to the magnetic field of the magnet 345, closing or opening a circuit within the sensor, thus, acting as a switch. Alternatively, the magnetic sensor 340 measures the magnetic field of the magnet 345 and determines if the magnet 345 is in close proximity based on the value of the measured magnetic field. In some embodiments, the housing 320 additionally includes a magnet or electromagnet that can be used to prevent the bins from opening by attracting the magnet 345 embedded within the bin. For example, a magnetic field of an electromagnet is controlled to attract the magnet 345 of the bin to lock the bin during a first time period. Additionally, the electromagnet is controlled to turn off or to repel the magnet 345 to unlock the bin during a second time period. The bin can be controlled to be unlocked during a time window corresponding to when the patient is scheduled to consume the contents of the bin, and to remain locked outside of that time window. In other embodiments, a mechanical component (e.g., spring) may be used to prevent the bins from being unintentionally dislodged from the outer housing. In some embodiments, the tactility of the mechanisms can provide a “spring/bounce” feeling to the user of the pillbox 125.

Referring back to FIG. 2, the tracking device 125 may include components to provide feedback or to provide notifications to the patient. For example, the housing or the bins of the pillbox 125 include one or more lighting elements (e.g., light emitting diodes or LED) to notify a patient to consume the contents of a specific bin. The housing of the pillbox 125 may include an LEDs corresponding to each bin that turn on or blinks when the patient is scheduled to consume the contents of the bin. The LED remains on or blinking until the patient opens the bin or a timeout occurs. In another example, the pillbox 125 includes an audio feedback element (such as a speaker or piezo electric speaker) to output audio signals to notify the patient to consume the contents of a bin. Moreover, the audio feedback element may be used to provide audio feedback to aid the patient to find the pillbox (e.g., when the pillbox was misplaced and the patient is unable to find the pillbox).

In another example, the tracking device is a dispenser other than a pillbox. For example, the dispenser 125 is a medication dispenser that stores and dispenses sealed packs (e.g., heat sealed packs) of pills. The dispenser 125 includes one or more sensors or determining whether a medication pack was dispensed to the patient. In another example, the dispenser 125 stores and dispenses liquids or aerosols. The dispenser 125 may track each time some or all of the contents stored therein are dispensed. Moreover, the dispenser 125 may track an amount that is being dispensed. Additionally, the dispenser may be able to determine other types of events, such as, when the dispenser was refilled. For instance, the dispenser may be a smart water bottle that is able to track the amount of water consumed by a patient. In other examples, the dispenser is a smart vaping device, a smart inhaler, or other similar devices.

In another example, the tracking device is a fitness tracker. The fitness tracker 125 includes sensors for determining a type of physical activity or an amount of physical activity performed by a patient. For example, the fitness tracker includes an accelerometer, a gyroscope, a pedometer, a global positioning system (GPS) receiver, a heart rate monitor, and a microphone. In some embodiments, the fitness tracker may be a wearable device (e.g., that can be worn around the wrist, ankle, head, neck or chest). In other embodiments, the fitness tracker is part of the client device 110. That is, the client device (such as a smartphone) may act as a fitness tracker by using one or more sensors embedded therein.

One or more third party systems 130 may be coupled to the network 150 for communicating with the health monitoring system 140, which is further described below in conjunction with FIG. 2. In one embodiment, a third-party system 130 is an application provider communicating information describing applications for execution by a client device 110 or communicating data to client devices 110 for use by an application executing on the client device. In other embodiments, a third-party system 130 provides content or other information for presentation via a client device 110. A third-party system 130 may also communicate information to the health monitoring system 140, such as advertisements, content, or information about an application provided by the third-party system 130.

In some embodiments, the third party systems 130 include entities storing electronic health records (EHR) for one or more patients of the health monitoring system. The EHR may be stored and shared using a pre-specified standard. In some embodiments, the EHR includes a collection of electronic health information of individual patients or populations. The EHR may include electronic medical records (EMR) that includes information created by providers for specific encounters in hospitals and ambulatory environments. Additionally, the EHR may include patient health records (PHR) that stores personal medical data generated or provided by individual patients themselves.

FIG. 2 is a block diagram of an architecture of the health monitoring system 140, in accordance with one or more embodiments. The health monitoring system 140 shown in FIG. 2 includes a user profile store 205, an analysis module 220, a data correlation module 230, a health recommendation module 240, and a web server 260. In other embodiments, the health monitoring system 140 may include additional, fewer, or different components for various applications. Conventional components such as network interfaces, security functions, load balancers, failover servers, management and network operations consoles, and the like are not shown so as to not obscure the details of the system architecture.

Each user of the health monitoring system 140 is associated with a user profile, which is stored in the user profile store 205. A user profile includes declarative information about the user that was explicitly shared by the user and may also include profile information inferred by the health monitoring system 140. In one embodiment, a user profile includes multiple data fields, each describing one or more attributes of the corresponding health monitoring system user. Examples of information stored in a user profile include biographic, demographic, and other types of descriptive information, such as work experience, educational history, gender, hobbies or preferences, location and the like. A user profile may also store other information provided by the user, for example, images or videos.

Users of the health monitoring system 140 include patients and healthcare/wellness professionals (e.g., medical doctors, nurses, nutritionists, wellness coaches, fitness coaches). For simplicity, both healthcare professionals and wellness professionals are referred to as “healthcare professionals” below. As such, throughout the description below, the various embodiments and examples that are provided for healthcare professional also apply to wellness professionals and any other type of professionals that may communicate with a patient (or user) of the health monitoring system 140 to provide or suggest one or more regimens for the patient.

For healthcare professionals, the user profile store stores a list of patients that are associated with the healthcare professional. In some embodiments, the healthcare professionals send requests to the health monitoring system 140 to associate a patient with the healthcare professional. In some embodiments, the association between the patient and the healthcare professional is established upon receiving a confirmation from the patient authorizing the association (e.g., authorizing patient information to be shared with the healthcare professional).

For patients, the user profile store stores data received from a tracking device 125 associated with the patient, and one or more measuring devices 120 associated with the user. In some embodiments, data is stored for a set amount of time only. In other embodiments, the health monitoring system 140 processes the data received from a tracking device 125 or a measuring device 120 prior to being stored. Additionally, the user profile store of a patient includes an identification of a caregiver of the patient. The caregiver may be a family member or any other person that is able to easily communicate with the patient. The health monitoring system is able to communicate with the caregiver as a backup or secondary means of communication.

The analysis module 220 receive data captured by one or more tracking devices 125 or one or more measuring devices 120 and analyzes the received data. The analysis module 220 generates adherence data for a set routine or regimen for the patient. The adherence data may be generated in the form of a time series having a set of adherence data points. The adherence data may be a time series of Boolean values. Each data point of the time series of Boolean values indicates whether the patient completed the routine or regimen for a set time period. Alternatively, the adherence data may be a time series of numerical values within a set range of values. For example, each data point in the time series indicates a percentage of completion of a routine or regimen for the set time period. In another example, each data point in the time series indicates an amount associated with the tracked routine or regimen (e.g., an amount of water consumed in a set time period, or an amount of physical activity exerted during a set time period).

For example, using sensor data captured by a pillbox 125, the analysis module 220 generates drug adherence data indicating how well a patient is adhering to a prescription regimen. Each data point in the time series for the drug adherence data indicates whether a patient consumed one or more medicine pills within a pre-specified time slot. As such, the drug adherence data is a time series of Boolean values. The pre-specified time slots may be configured by a healthcare professional (e.g., a primary care physician) of a patient. Alternatively, the pre-specified time slopes are configured by the patients themselves using the client device 110. In some embodiments, the analysis module 220 additionally determines an adherence rate indicating how often the patient consumed the one or more pills as prescribed by the healthcare professional.

In another example, using data captured by a fitness tracker, the analysis module 220 generates physical activity adherence data indicating an amount of exercise performed for each time period being tracked. The physical activity adherence data is a time series of numerical values, each indicating an amount of exercised performed by the patient in a set time period. The amount of exercise may be measured based on the amount of time the patient spent exercising each time period, or based on the number of calories burned by the patient during each time period.

Other types of adherence data that may be tracked include water consumption, alcohol consumption, food consumption (including meal logging, and tracking of calories, carbohydrates, protein, fat, and fiber consumption), meditation time, sun exposure, and the like.

The analysis module 220 also generates physiological data for patients based on measurements captured by one or more measuring devices of each patient. The physiological data for a patient may be generated in the form of a time series having a set of physiological data points. In some embodiments, the analysis module 220 identifies trends in a patient's physiological data. For example, the analysis module 220 identifies whether the patient's physiological data is increasing or decreasing. Moreover, the analysis module 220 identifies whether the patient's physiological data is within an expected range (e.g., a healthy range). The expected range may be provided to the analysis module 220. For example, the analysis module 220 may be configured to consider a first range of blood pressure levels as being a healthy range, a second range as being an elevated range, and a third range as being an unhealthy range. In some embodiments, the analysis module 220 learn the expected ranges based on physiological data provided by a large number of patients. For example, the analysis module 220 may determine an average blood pressure level for a population based on physiological data provided by members of the population. In some embodiments, the analysis module 220 uses profile information (such as age, height, and weight of a patient) in determining an expected range for a particular type of physiological data.

In some embodiments, the analysis module 220 additionally generates physiological data based on data manually inputted by the patient. For example, users may provide information about their mood or their level of pain for a given time period. In yet other embodiments, the analysis module 220 generates physiological data based on photos captured by the client device 110 of a patient or provided by the patient to the health monitoring system 140. The analysis module 220 applies a classifier module to the photo to generate the physiological data. For example, the analysis module 220 determines a mood of the patient based on a selfie taken by the patient. In another example, the analysis module 220 determines a severity of a skin condition by applying a classifier module to a picture showing the affected skin area of the user.

The data correlation module 230 receives physiological data for a patient and adherence data (e.g., drug adherence data or physical activity adherence data) for the patient, and performs a correlation analysis between the patient's physiological data and the patient's adherence data. For example, the data correlation module performs a correlation analysis between the drug adherence data for a patient and the physiological data for the patient. In another example, the data correlation module 230 correlates a patient's physical activity data with the patient's physiological data.

The data correlation module 230 generates a score indicative of the correlation between one type of data and a second type of data. In particular, the data correlation module 230 generates a score indicative of the correlation between adherence data (e.g., drug adherence data or physical activity adherence data) and physiological data for a patient. For example, the score may indicate a level of correlation between the two types of data. For instance, the data correlation module 240 increases the correlation score when the physiological data improves when the adherence data indicates the patient consumed the prescribed medicine, and decreases the correlation score when the physiological data worsens when the adherence data indicates the patient consumed the prescribed medicine.

In some embodiments, when the adherence data is a numerical value (i.e., when the adherence data indicates an amount associated with a specific regimen, such as, an amount of water consumed, an amount of alcohol consumed, or an amount of exercises performed), the data correlation module 230 determines a correlation between the amount associated with a specific regimen and a change in the patient's physiological data. For example, when the adherence data is a physical activity adherence data, the data correlation module 230 determines a correlation between an amount of physical activity performed by the patient to an amount of change in the patient's physiological data.

In some embodiments, the data correlation module 230 performed the analysis using a trained model. The data correlation module 230 provides each of the adherence data and the physiological data as time series for the patient, and the trained model outputs one or more numerical results (e.g., a correlation score). The numerical scores can then be used to provide recommendations to users of the health monitoring system 140.

The health recommendation module 240 provides recommendations based on the output of the data correlation module 230. The health recommendation module 240 may provide recommendations directly to a patient based on the analysis of the patient's adherence and physiological data. Alternatively, the health recommendation module 240 provides recommendations or insights to a healthcare professional regarding a patient associated with the healthcare professional.

The web server 260 links the health monitoring system 140 via the network 150 to the one or more client devices 110, as well as to the one or more third party systems 130. The web server 260 serves web pages, as well as other content, such as JAVA®, FLASH®, XML and so forth. The web server 260 may receive and route messages between the health monitoring system 140 and the client device 110, for example, instant messages, queued messages (e.g., email), text messages, short message service (SMS) messages, or messages sent using any other suitable messaging technique. A user may send a request to the web server 260 to upload information (e.g., images or videos) that are stored in a content store or in the user profile store. Additionally, the web server 260 may provide application programming interface (API) functionality to send data directly to native client device operating systems, such as IOS®, ANDROID™, or BlackberryOS.

### Health Monitoring System

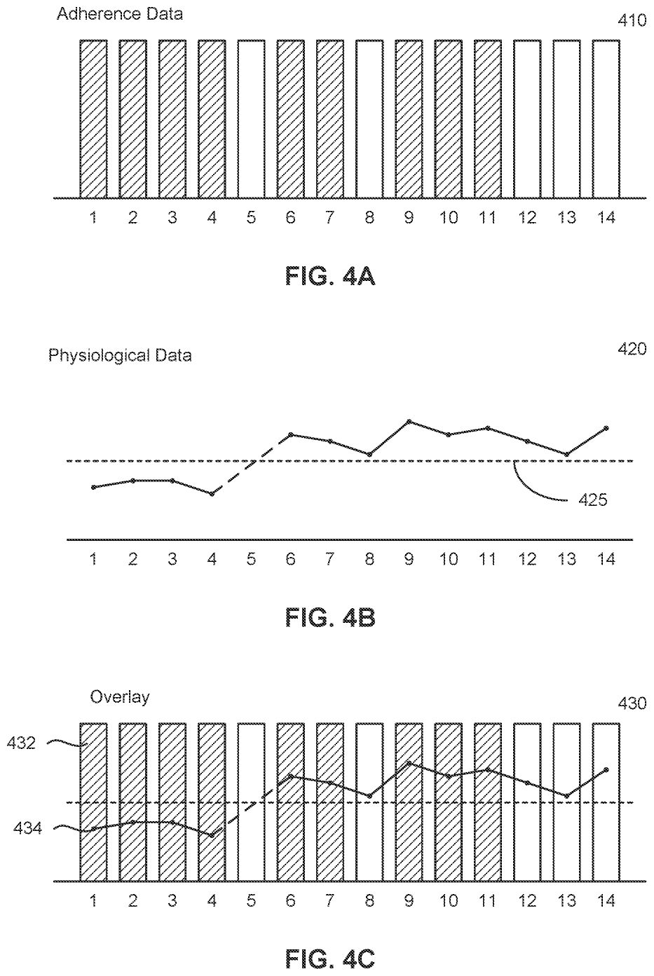

The health monitoring system 140 provides patient information to a patient, a healthcare professional assigned to the patient, or a caregiver of a patient. Based on the sensor data captured by a pillbox 125, the health monitoring system 140 provides a patient's drug adherence data. For example, FIG. 4A shows a graph identifying whether a patient took a particular medicine pill. For each day of a set of days (e.g., past 2-week period), the graph 410 shows whether the patient took one or more medicine/supplements pills within a specified time window. For example, graph 410 indicates that the patient took the one or more medicine pills on time on days 1-4, 6-7 and 9-11, and did not take the medicine pills on days 5, 8, and 12-14. In some embodiments, the graph 410 further identifies if the patient took a medicine pill outside of the specified time window (e.g., if the patient took a medicine pill early or late). For instance, the graph 410 may use a first color (e.g., green) to indicate that the patient took the medicine pill on time in a given day, a second color (e.g., red) to indicate that the patient did not take the medicine pill on a given day, or third color (e.g., yellow) to indicate that the patient took the medicine pill outside of the specified time window. In some embodiments, the graph 410 uses a color gradient (e.g., between green and red) to indicate how late was the patient in taking the medicine pill.

Based on the data captured by the one or more measuring devices 120, the health monitoring system 140 provides a patient's physiological data. For example, FIG. 4B shows a graph plotting the physiological data (e.g., the patient's glucose level) as function of time. In some embodiments, the graph 420 identifies days for which physiological data for the patient is not available (e.g., physiological data for the patient was not received from one or more measuring devices 120). For example, in the graph 420 of FIG. 4B, physiological data for day 5 is unavailable. The graph 420 indicates that the physiological data for day 5 is unavailable by adding a dashed line between the physiological data for day 4 and the physiological data for day 6. In other embodiments, other ways of indicating that physiological data for specific dates are possible (e.g., by using different color lines or different shape markers). Moreover, the graph 420 may additionally indicate the time of day the physiological data was taken. For example, the graph may use a marker with a first shape (e.g., square) or a first color (e.g., green) if the physiological data was taken during a first time period (e.g., morning), a second shape (e.g., circle) or a second color (e.g., blue) if the physiological data was taken during a second time period (e.g., afternoon), and so on.

In some embodiments, the graph 420 additionally provides an indication of a target level 425. The target level 425 may be a threshold level. For example, the target level 425 indicates the boundary between a healthy or recommended range for the physiological data (e.g., a healthy or recommended blood glucose level range). The target level 425 may be determined based on a set of guidelines. Additionally, the target level 425 may be determined based on information from the patient's profile (e.g., weight, height, or age).

The health monitoring system 140 further presents a graph overlaying the patient's drug adherence data with the patient's physiological data. For example, FIG. 4C shows a graph 430 plotting a patient's drug adherence data and physiological data combined into a single graph. The graph 430 is divided into a set of time periods (e.g., each corresponding to one day). For each time period, the graph identifies whether the patient consumed the medicine pill (as indicated by bars 432) and plots the physiological data point corresponding to the time period (as identified by point 434). Using the overlaid graph 430, a healthcare professional is able to get insights on the efficacy of a medication prescribed to a patient. For example, from the overlaid graph 430, a healthcare professional is able to see a correlation between the consumption of the prescribed medication and the physiological data. From the overlaid graph 430, the healthcare professional may identify whether the physiological data trends in a first direction when the patient consumed the prescribed medication as prescribed, whether the physiological data trends in a second direction when the patient failed to consume the prescribed medication as prescribed, or whether the physiological data does not follow a noticeable correlation with the patient's drug adherence data.

Based on the adherence graph 410, the physiological graph 420 and the overlaid graph 430, a healthcare professional is able to better understand a patient's response to a specific medication and adjust the prescription regimen accordingly. For example, if the adherence graph 410 indicates that the patient has a high adherence rate (i.e., the patient regularly takes the prescribed medication on time) but the overlaid graph 430 does not show an appreciable improvement in physiological data when the patient took the medication as prescribed, the healthcare professional may decide to make changes to the prescription regimen (e.g., by changing the dose or frequency of the prescribed medication, or changing the medication altogether).

Alternatively, if the adherence graph 410 indicates that the patient has a low adherence rate but the overlaid graph 430 shows an improvement in physiological data whenever the patient takes the medication as prescribed, the healthcare professional may make changes to improve the patient adherence. For example, the healthcare professional may adjust the health monitoring system 140 to provide more frequent reminders to the patient to take the prescribed medication, or may configure the health monitoring system 140 provide automated reminders to a caregiver (such as a family member of the patient) to encourage the caregiver to remind the patient to take the prescribed medication as scheduled. Moreover, the healthcare professional may be able to further inquire the patient regarding the reasons for the low adherence rate. Based on the discussion between the healthcare professional and the patient, the healthcare professional may be able to further modify the prescription regimen. For example, if the patient indicates that it is hard due to certain circumstances to take the prescribed medicine during specific time windows, the healthcare professional is able to adjust the medication schedule accordingly. Alternatively, if the patient indicates that the low adherence rate is due to secondary effects of the prescribed medication that are not reflected in the data captured by the health monitoring system 140, the healthcare professional is able to adjust the dose or change the medication to reduce the undesirable secondary effects.

In some embodiments, based on the patient's drug adherence data and the patient's physiological data, the health monitoring system 140 determines one or more scores for the patient. For example, the health monitoring system 140 determines a score based on a correlation between the patient's drug adherence data and the patient's physiological data. For example, the health monitoring system 140 may determine if the physiological data for the patient improves (e.g., gets within or closer to an acceptable range). For each data point in the physiological data, the health monitoring system 140 may determine whether the compliance data shows that the patient took a medication as scheduled prior to when the physiological data was taken. Based on the information regarding whether the patient took the medication as scheduled prior to when the physiological data was taken, and the physiological data itself or a change in physiological data (e.g., the difference between the patient's physiological data before the medication was taken and after the medication was taken), the health monitoring system 140 modifies a correlation score for the patient. Moreover, the correlation score may be modified based on a adherence rate or an average for the physiological data for a predetermined period of time. In some embodiments, the correlation score is determined using a trained model generated using training data including compliance data and physiological data of a set of patients.

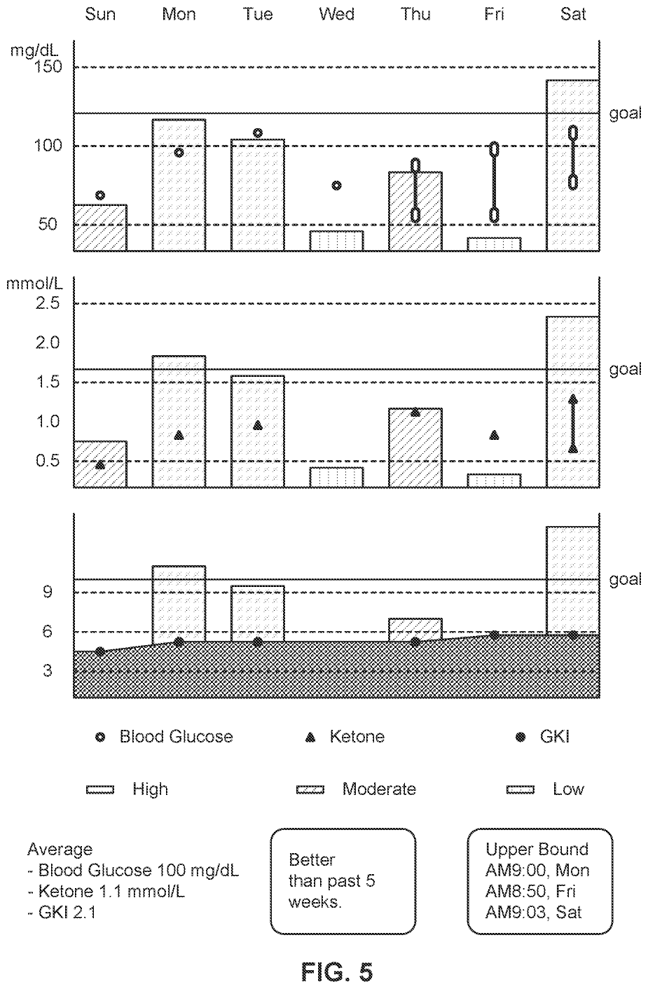

In some embodiments, the health monitoring system 140 correlates other types of adherence data with physiological data captured by one or more measurement devices 120. For example, the health monitoring system 140 correlates physical activity adherence data with physiological data for a patient. FIG. 5 shows graphs overlaying a patient's physical activity adherence data with a patient's physiological data, in accordance with one or more embodiments. In particular, the graphs shown in FIG. 5 overlay a patient's physical adherence activity data with the patient's blood glucose level, ketone level, and glucose ketone index (GKI) over the span of a week. The physical activity adherence data may be determined based on data captured by the client device 110, a measurement device 120 (such as a fitness tracker), or may be entered manually by a patient. In some embodiments, a patient's physical activity is measured based on the amount of time the patient spent exercising. Alternatively, a patient's physical activity is measured based on a number of calories burned by a patient in a given day. The health monitoring system 140 may classify the patient's physical activity into high, moderate, and low. The classification may be based on the patient's preference, or may be based on the patient's profile (e.g., based on the patient's age, height, and weight).

The health monitoring system 140 may perform a correlation analysis between the patient's physical activity adherence data and the patient's physiological data. For example, the health monitoring system 140 may determine a trend in the patient's physiological data as a function of physical activity. Based on the analysis, the health monitoring system 140 may determine a suggested level of exercise to enable the patient to achieve a desired level in the patient's physiological data.

In some embodiments, the health monitoring system 140 allows the patient to specify a type of exercise conducted throughout a given time period. For example, a patient may specify that the exercise performed included a 30-minute cardio session. The health monitoring system 140 can then use the information regarding the type of exercise performed in the correlation analysis to provide the patient a more tailored recommendation. For example, the health monitoring system 140 may provide a recommendation to perform a combination of different types of exercises to enable the user to achieve a desired level in the patient's physiological data. The health monitoring system may provide various exercise combination for the patient to choose based on the patient's preference. For example, the health monitoring system 140 may recommend a patient to have a 30-minute cardio session, or a 15-minute cardio session followed by a 45-minute of free weight training. Alternatively, the health monitoring system 140 may inform the patient of types of exercises that do not seem to result in improvement in the patient's physiological data.

In some embodiments, the health monitoring system 140 performs a correlation analysis using the patient's drug adherence data, the patient's physical activity adherence data, and the patient's physiological data. Using the correlation analysis, the health monitoring system 140 may be able to provide insights on how the medication being consumed by the patient interacts with physical activity by the patient to affect the patient's physiological data.

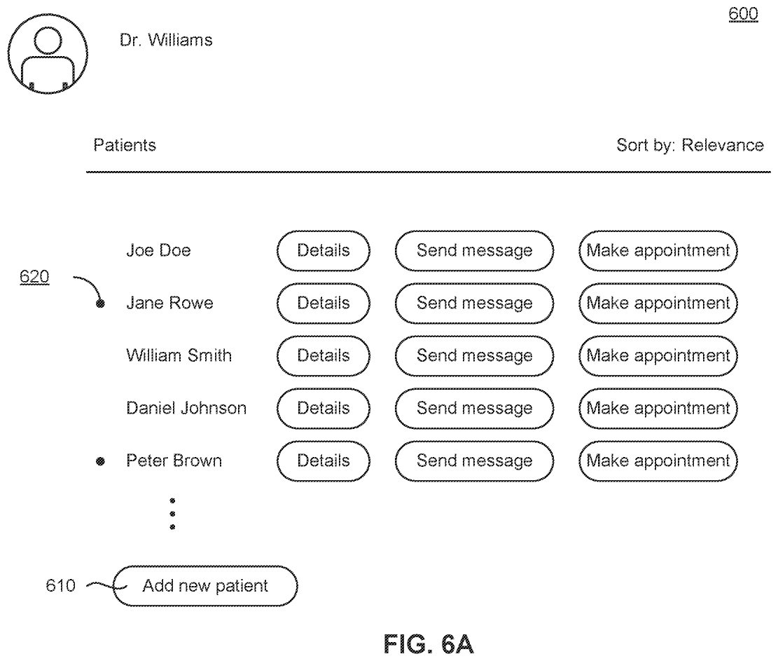

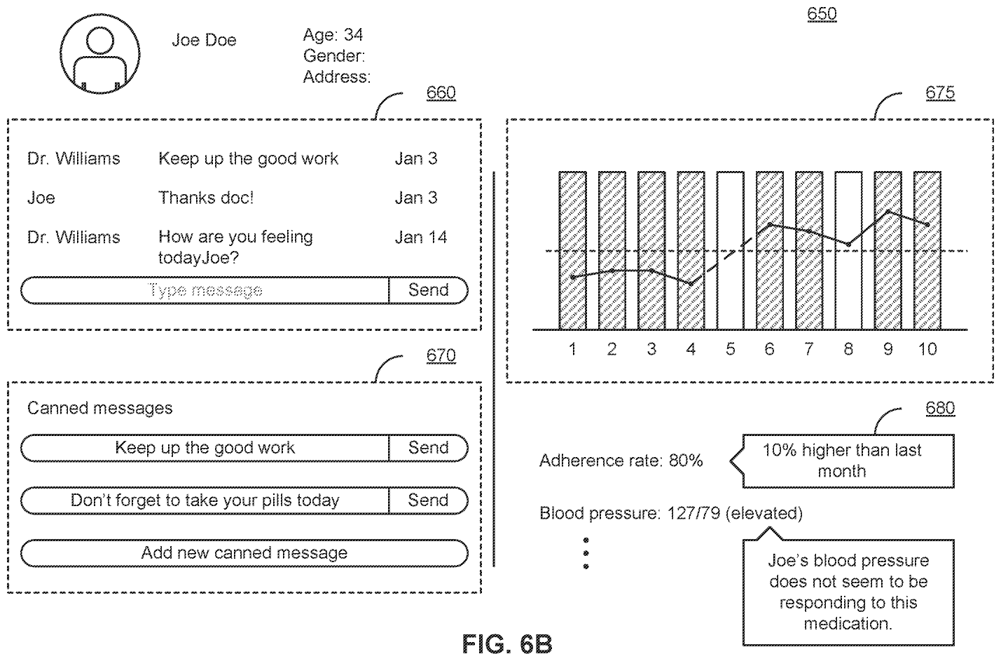

The health monitoring system 140 enables healthcare professionals to access information for each of their patients and to quickly identify patients that would benefit from additional attention by the healthcare professional. FIG. 6A illustrates a user interface 600 showing a list of patients for a healthcare professional in the health monitoring system 140, according to one embodiment. The user interface 600 provides a list of patients that are registered under the healthcare professional. For example, the healthcare professional may be primary care physician (PCP) of the patients listed in the user interface 600. The healthcare professional may be able to enroll new patients into the health monitoring system 140. For example, the user interface 600 provides a user interface element 610 (e.g., a button) to link the healthcare professional to an enrollment page (not show). In the enrollment page, the healthcare professional may be able to enter patient information to complete the enrollment process. In some embodiments, the enrollment page causes an invitation to be sent to the patient being enrolled (e.g., to an email address provided by the healthcare professional). In some embodiments, during the enrollment process, a client device 110 for the patient may be associated with a user profile of the patient. Additionally, a pillbox 125 or one or more measuring devices 120 may be associated with the user profile of the patient.

The user interface 600 sorts the patients based one or more sorting criteria. For example, the patients are sorted based on a correlation score determined based on each patient's drug adherence data and physiological data. In some embodiments, patients are sorted based on a relevance score determined based on the correlation score and other information corresponding to each user. By sorting the patients using the compliance score or a relevance scored determined based on the compliance score, a healthcare profession may gain insight on which of his or her patients show a low correlation between their compliance in adhering to a prescription and their physiological data. This way, the healthcare professional can schedule a follow up appointment with the patients that show low correlation to adjust their prescription.

Additionally, the user interface 600 can sort the patients by a compliance rate. The user interface 600 may show patients with low compliance rates before patients with higher compliance rates. As such, the healthcare professional is able to identify patients that are not adhering to their prescriptions to encourage them to take their medicine as recommended by the healthcare professional. Additionally, the healthcare professional may be able schedule follow up appointments with patients that have low compliance rates to discuss how they can improve their adherence rate.

In some embodiments, the user interface 600 identifies patients that need attention. For example, the user interface 600 indicates, using a user interface element 620, that a patient has provided a new message to the healthcare professional that the healthcare professional has not read yet. In some embodiments, the user interface 600 allows the healthcare professional to sort or filter patients based on whether an action by the healthcare professional is needed for the patient, or based on whether the patient needs attention by the healthcare professional.

In some embodiments, the user interface 600 allows healthcare professionals to schedule or perform virtual appointments. The healthcare professional may be able to send and receive messages to interact with a patient. Additionally, the user interface 600 may allow the healthcare professional to conduct a video conference or a phone call to interact with a patient in real-time. The health monitoring system 140 may allow the healthcare professional to get physiological data measured by a patient's measuring device 120 in real-time during the course of a virtual appointment. For instance, the healthcare professional can instruct the patient to activate a specific measuring device 120 that is connected to the client device 110 of the user or is connected directly to the health monitoring system 140 through the network. In some embodiments, the healthcare professional may be able to control the measuring device of a patient through the health monitoring system 140. For example, the healthcare professional may be able to adjust settings of the measuring device 120 or to instruct the measuring device 120 to start or stop recording measurements.

In some embodiments, a healthcare professional can send a package with additional measuring devices to a patient prior to a scheduled virtual appointment. The additional measuring devices may be pre-configured to connect to the health monitoring system 140 before they are sent to the patient. Moreover, the additional measuring devices may be pre-configured to associate recorded data with a user account of a specific patient prior to being sent to the patient. In some embodiments, the additional measuring devices are sent by the health monitoring system 140 in response to the scheduling of a virtual appointment by a healthcare professional. That is, when the healthcare professional schedules a virtual appointment with a patient, the health monitoring system 140 receives a request to send one or more measuring devices to the patient. The type of the additional measuring devices sent to the patient may be based a type of virtual appointment, or may be specified by the healthcare professional when scheduling the virtual appointment. In some embodiments, the package sent to the patient includes a return label to allow the patient to return the additional measuring devices to the healthcare professional or the health monitoring system. Moreover, the additional measuring devices may be disabled while they are in transit to or from the patient's residence or outside of the time window of the virtual appointment.

FIG. 6B illustrates a user interface 650 showing details for a patient of the health monitoring system 140, according to one embodiment. The user interface 650 includes a message thread 660. The message thread includes messages provided from the patient to the healthcare professional, or messages provided from the healthcare professional to the patient. For example, the message thread 660 of FIG. 6B includes a message from Dr. Williams to the patient provided on January 3, and a response from the patient to the doctor's message. The

The message thread 660 allows for quick and informal conversations between a patient and a healthcare professional. The message thread 660 can be used by a healthcare professional to encourage a patient to keep adhering to a prescription regimen. In some embodiments, the healthcare professional can select one of a set of template messages to send to the patient. In some embodiments, the template messages are saved by the healthcare professional. In other embodiments, the canned responses are suggested by the health monitoring system 140 to the healthcare professional. For example, the health monitoring system provides suggested canned based on the patient's drug adherence data and physiological data. If the patient's drug adherence data shows a low adherence rate, the template messages may include messages to encourage the patient to increase his or her adherence rate. For example, template messages include messages such as “don't forget to take your pills today.” Alternatively, if the drug adherence data shows a high adherence rate, the template messages may include messages for praising the patient for the high adherence rate. For example, template messages include messages such as “keep up the good work.”

In some embodiments, the template messages include messages for inquiring a patient for updates. For example, the template messages include messages such as “how are you feeling today?” In some embodiments, some template messages are suggested to the healthcare professional in response to a substantial change in a patient's drug adherence data or physiological data. For example, if a user's physiological data changes by an amount that is larger than a set threshold value, the health monitoring system 140 suggests the healthcare professional to inquire the patient about how the patient is feeling.

In some embodiments, the template messages are suggested using a trained mode. The model may be trained using passed messages sent by healthcare professionals to a set of patients. For example, the training module determines whether a message is a commonly sent message. For commonly sent messages, the training module trains a model based on a patient's drug adherence data and physiological data when the message was sent to determine when to suggest the message. In some embodiments, the patient identifiable information is anonymized prior to providing the training data to the training module to protect the patient's privacy. For example, if a message included in the training data contains the patient's name, the patient's name is removed prior to using the message for training a model for suggesting template messages.

In some embodiments, the user interface 650 additionally includes insights 680 determined based on the patient's drug adherence data and physiological data. The insights 680 include a patient's adherence rate, changes in the patient's adherence rate, patient's average physiological data, an indication of whether the patient's physiological data is within an expected range. In some embodiments, the user interface 650 provides suggested actions for a healthcare professional based one or more insights. For example, if an insight based on a patient's physiological data shows that the patient's physiological data is not improving as expected, the health monitoring system 140 suggests the healthcare professional to schedule an appointment with the patient to re-evaluate the prescription regimen.

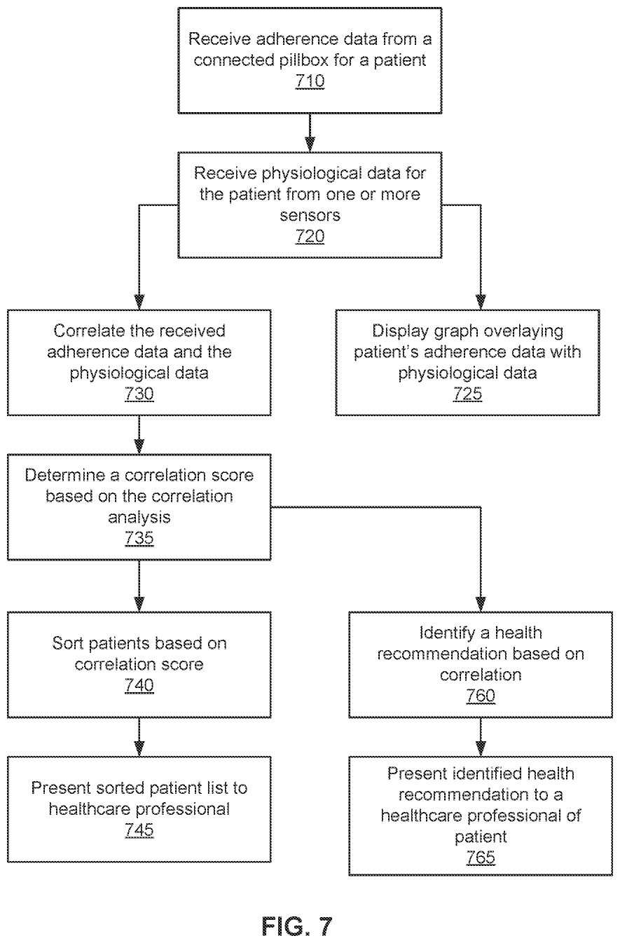

FIG. 7 illustrates a flow diagram for employing patient's adherence data (such as drug adherence data or physical activity adherence data) and patient's physiological data in the remote health monitoring of a patient, according to one embodiment. The health monitoring system 140 receives 710 adherence data for one or more patients. For example, drug adherence data for a patient may be received from a connected pillbox 125 associated with a user account of the patient, or may be derived from sensor data captured by one or more sensors of the connected pillbox 125 associated with the user account of the patient. In another example, physical activity adherence data is received from a fitness tracker associated with a patient. Additionally, the health monitoring system 140 receives 720 physiological data for the one or more patients. The physiological data for a patient may be received from one or more measuring devices 120 associated with the user account of the patient. Alternatively, the physiological data for the patient may be received from a client device 110 associated with the user account of the patient. In this embodiment, the client device of the patient receives the physiological data from one or more measuring devices connected to the client device, and the client device transmits the physiological data to the health monitoring system 140. In some embodiments, the client device has one or more measuring devices to capture certain types of physiological data. For example, the client device is able to capture heart rate data using a heart rate sensor. In yet other embodiments, the patient is able to manually enter measurement captured using measuring devices that do not have capabilities for being connected to the client device or the network.

In some embodiments, the health monitoring system 140 presents the adherence data and the physiological data for a patient to a healthcare professional to aid the healthcare professional in determining the efficacy of the drug prescription regimen for the patient. For example, a graph showing the drug adherence data overlaid with the physiological data for the patient during a set timeframe is displayed 725 to the healthcare professional to show the change in physiological data for the patient to the healthcare professional. In another example, a graph showing physical activity adherence data overlaid with physiological data for the patient during a set timeframe is displayed 725 to the patient to show a correlation between physical activity and an improvement in physiological data.

In some embodiments, the health monitoring system 140 correlates 730 the adherence data of a patient and the physiological data of the patient. The health monitoring system 140 may apply a trained model to the adherence data of the patient and the physiological data of the patient to determine 735 a correlation score.

The health monitoring system 140 uses the correlation score or other information obtained as a result of the correlation analysis between the patient's adherence data and the patient's physiological data for various purposes. For example, the health monitoring system 140 sorts 740 a list of patients for a healthcare professional based on the correlation score and presents 745 the sorted list of patients to the healthcare professional. As such, the healthcare professional is able to identify patients with low correlation between their drug adherence and their physiological data to determine whether changes to their drug prescription is desired. Alternatively, the health monitoring system 140 identifies 760 one or more health recommendations based on the correlation between the patient's drug adherence data and the patient's physiological data and presents 765 the identified health recommendations to a healthcare professional of the patient. The health recommendations include recommendations for the healthcare professional to evaluate whether certain actions are desirable. For example, the health recommendations include suggestions to send messages (e.g., template messages) to a patient, to schedule an appointment (e.g., a virtual appointment) with the patient, or to revise or change the prescription regimen of the patient. The health recommendations additionally include recommendations for the patients themselves. For example, the recommendations include suggestions to contact the patient's healthcare professional to schedule a follow up appointment, to perform certain a activities such as physical exercises, breathing exercises, or meditation, or to consume certain foods or supplements. Additionally, the health recommendations may include recommendations for other parties associated with the patient. For example, the health recommendations include recommendations for a caregiver of the patient (e.g., a family member assigned as the caregiver of the patient within the health monitoring system). The recommendations include suggestions to contact the patient to remind the patient to consume a prescribed drug as scheduled.

### CONCLUSION

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
receiving sensor data from a tracking device associated with a patient of a health monitoring system;
generating adherence data based on the received sensor data, the adherence data including a set of adherence data points, each adherence data point corresponding to a time period of a set of time periods;
receiving physiological data from one or more measuring devices associated with the patient of the health monitoring system, the physiological data including a set of physiological data points, each physiological data point corresponding to a time period of the set of time periods; and
providing, to a user of the health monitoring system, a user interface element generated based on a correlation between the generated adherence data and the physiological data.

2. The method of claim 1, wherein the tracking device is a pillbox having a plurality of sensors for determining whether a compartment of the pillbox has been opened by the patient, and wherein the adherence data is a drug adherence data indicating whether the patient consumed a prescribed medication within a set time window.

3. The method of claim 1, wherein the tracking device is a fitness tracker for tracking an amount of physical activity performed by the patient, and wherein the adherence data is a physical activity adherence data indicating an amount of physical activity performed by the patient during each time period of the set of time periods.

4. The method of claim 1, wherein the one or more measuring devices include at least one of a sphygmomanometer, a glucometer, a thermometer, a pulse oximeter, an electrocardiogram (ECG/EKG) monitor, and a breath analyzer.

5. The method of claim 1, wherein the tracking device and the one or more measuring devices are configured to be connected to a client device of the patient, and wherein the sensor data captured by the tracking device and the physiological data captured by the one or more measuring devices are received by the health monitoring system from the client device of the patient.

6. The method of claim 1, wherein the user interface element is a graph for presenting the correlation between the generated adherence data and the physiological data for the patient.

7. The method of claim 4, wherein the graph overlays the adherence data with the physiological data, wherein the graph is divided into a plurality of time periods, wherein each time period of the plurality of time periods displays a corresponding adherence data point overlaid with a corresponding physiological data point.

8. The method of claim 1, wherein the user interface element is a set of recommendations provided to a healthcare professional associated with the patient, wherein each recommendation of the set of recommendations is identified by applying a trained model to the generated adherence data and the physiological data for the patient.

9. The method of claim 1, wherein the user interface element is a list of template messages for sending to the patient, wherein each template message of the list of template messages is selected by applying a trained model to the generated adherence data and the physiological data for the patient.

10. The method of claim 1, wherein the user interface element is a list of patients associated with a healthcare professional, and wherein the method further comprises:
for each patient of a plurality of patients associated with the healthcare professional, determining a relevance score based on a correlation analysis between the generated adherence data for the patient and the physiological data for the patient; and
sorting the list of patients associated with the healthcare professional based on the determined relevance score.

11. A non-transitory computer-readable storage medium configured to store instructions, the instructions when executed by a processor cause the processor to:
receive sensor data from a tracking device associated with a patient of a health monitoring system;
generate adherence data based on the received sensor data, the adherence data including a set of adherence data points, each adherence data point corresponding to a time period of a set of time periods;
receive physiological data from one or more measuring devices associated with the patient of the health monitoring system, the physiological data including a set of physiological data points, each physiological data point corresponding to a time period of the set of time periods; and
provide, to a user of the health monitoring system, a user interface element generated based on a correlation between the generated adherence data and the physiological data.

12. The non-transitory computer-readable storage medium of claim 11, wherein the tracking device is a pillbox having a plurality of sensors for determining whether a compartment of the pillbox has been opened by the patient, and wherein the adherence data is a drug adherence data indicating whether the patient consumed a prescribed medication within a set time window.

13. The non-transitory computer-readable storage medium of claim 11, wherein the tracking device is a fitness tracker for tracking an amount of physical activity performed by the patient, and wherein the adherence data is a physical activity adherence data indicating an amount of physical activity performed by the patient during each time period of the set of time periods.

14. The non-transitory computer-readable storage medium of claim 11, wherein the one or more measuring devices include at least one of a sphygmomanometer, a glucometer, a thermometer, a pulse oximeter, an electrocardiogram (ECG/EKG) monitor, and a breath analyzer.

15. The non-transitory computer-readable storage medium of claim 11, wherein the tracking device and the one or more measuring devices are configured to be connected to a client device of the patient, and wherein the sensor data captured by the tracking device and the physiological data captured by the one or more measuring devices are received by the health monitoring system from the client device of the patient.

16. The non-transitory computer-readable storage medium of claim 11, wherein the user interface element is a graph for presenting the correlation between the generated adherence data and the physiological data for the patient.

17. The non-transitory computer-readable storage medium of claim 14, wherein the graph overlays the adherence data with the physiological data, wherein the graph is divided into a plurality of time periods, wherein each time period of the plurality of time periods displays a corresponding adherence data point overlaid with a corresponding physiological data point.

18. The non-transitory computer-readable storage medium of claim 11, wherein the user interface element is a set of recommendations provided to a healthcare professional associated with the patient, wherein each recommendation of the set of recommendations is identified by applying a trained model to the generated adherence data and the physiological data for the patient.

19. The non-transitory computer-readable storage medium of claim 11, wherein the user interface element is a list of template messages for sending to the patient, wherein each template message of the list of template messages is selected by applying a trained model to the generated adherence data and the physiological data for the patient.

20. The non-transitory computer-readable storage medium of claim 11, wherein the user interface element is a list of patients associated with a healthcare professional, and wherein the instructions further cause the processor to:
for each patient of a plurality of patients associated with the healthcare professional, determine a relevance score based on a correlation analysis between the generated adherence data for the patient and the physiological data for the patient; and
sort the list of patients associated with the healthcare professional based on the determined relevance score.
